(19) United States
(12) Patent Application Publication
(10) Pub. No.: US 2015/0160195 A1
(43) Pub. Date: Jun. 11, 2015

(54) HUMAN LIVER TUMOR CELL LINE AND METHOD OF AGENT SCREENING

(71) Applicant: Industrial Technology Research Institute, Hsinchu (TW)

(72) Inventors: Mei-Wei Lin, Hsinchu County (TW); Dian-Kun Li, Chiayi County (TW); Ling-Mei Wang, Taipei City (TW); Ching-Huai Ko, Changhua County (TW); Chin-Pen Lai, Hsinchu County (TW); Chun-Chung Wang, Kaohsiung City (TW); Hsiang-Wen Tseng, New Taipei City (TW)

(21) Appl. No.: 14/513,174

(22) Filed: Oct. 13, 2014

(30) Foreign Application Priority Data
Dec. 11, 2013 (TW) 102145618

(51) Int. Cl. G01N 33/50; C12N 5/071

(52) U.S. Cl. CPC G01N 33/5067; C12N 5/067; G01N 33/5011

(57) ABSTRACT

An isolated human liver tumor cell line is provided and is named as ITRI-H16, and which was deposited in the Food Industry Research and Development Institute with the accession number BCRC960432 on Nov. 7, 2011. A method of an agent screening is also provided. The method includes providing the isolated human liver tumor cell line ITRI-H16 and treating an interest compound to the ITRI-H16 cell line to determine an effect of the interesting compound on the ITRI-H16 cell line.

### CROSS-REFERENCE TO RELATED APPLICATION

This application claims the priority benefit of Taiwan application serial no. 102145618, filed on Dec. 11, 2013. The entirety of the above-mentioned patent application is hereby incorporated by reference herein and made a part of this specification.

### TECHNICAL FIELD

The technical field relates to an isolated human liver tumor cell line and a method of an agent screening.

### BACKGROUND

Compared with treatments of other cancers, a response rate of liver cancer to a traditional chemotherapy, such as doxorubicin and cisplatin, is less than 20%. Therefore, the treatment of liver cancer is very limited and is yet to be confirmed in clinical trials to extend a patient survival time by traditional chemical drugs. Further, a targeted drug of liver cancer, Sorafenib (Nexavar), approved by the U.S. Food and Drug Administration in 2007 only extends the patient survival time of three-month in phase III of human clinical trials. Hence, a drug having efficacy in the liver cancer treatment urgently needs to be developed.

Pathogenesis of liver cancer is quite complex, the main causes are inflections of hepatitis B virus (HBV) and hepatitis C virus (HCV), aflatoxin, alcohol, and other possible causes of cirrhosis. Hepatitis C is considered as the most main cause of liver cancer in the countries of United States, Japan, etc. Therefore, with the increased incidence of liver cancer worldwide each year, studies of the pathogenesis of HCV-related hepatocellular carcinoma (HCV-related HCC) and developments of HCV-related HCC drugs are urgent issues. However, with an overview of liver tumor cell lines preserved in the internationally renowned cell repository centers, the American Type Culture Collection (ATCC) and Japanese Collection of Research Bioresources (JCRB), most of the liver tumor cell lines are HBV-related or HBV-negative. A successful establishment of a liver tumor cell line derived from a liver tumor tissue of a HCV-related HCC patient is not yet achieved. Therefore, HCV-related liver tumor cell lines are urgently needed in this field for research so as to facilitate the developments of the HCV-related HCC drugs by testing and screening, which can make up a deficiency of a development platform of the liver cancer drugs.

### SUMMARY

The disclosure relates to an isolated human liver tumor cell line, which is capable of applying in researches of human HCV-related hepatocellular carcinoma (HCV-related HCC).

The disclosure relates to a method of an agent screening, which uses an isolated human liver tumor cell line to perform the agent screening, thereby developing drugs related to liver cancer, liver failure, or liver cancer stem cell.

An isolated human liver tumor cell line of the disclosure is named as ITRI-H16. The ITRI-H16 cell line was deposited in the Food Industry Research and Development Institute with an accession number BCRC960432 on Nov. 7, 2011.

A method of an agent screening of the disclosure includes the following steps. First, an isolated human liver tumor cell line is provided, which is named as ITRI-H16. The ITRI-H16 cell line was deposited in the Food Industry Research and Development Institute with an accession number BCRC960432 on Nov. 7, 2011. Next, an interesting compound is added into the isolated human liver tumor cell line so as to determine an effect of the interesting compound on the isolated human liver tumor cell line.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1A to FIG. 1C reveal a morphology of a monolayer-cell of a human liver tumor cell line ITRI-H16, which is observed under a phase contrast microscope with magnifications of 40×, 100×, and 200×, respectively according to one embodiment of the invention.

FIG. 2 shows a growth curve of a human liver tumor cell line ITRI-H16 according to one embodiment of the invention.

FIG. 3 shows a CYP3A4 activity of a human liver tumor cell line ITRI-H16 in an experiment group, a stimulator treatment group, an inhibitor treatment group, and a control group according to one embodiment of the invention.

FIG. 4 shows a cell viability of the human liver tumor cell line ITRI-H16 in the groups after performing treatments of Sorafenib in different concentrations according to one embodiment of the invention.

FIG. 5 shows a luciferase expression of a human liver tumor cell line ITRI-H16 transfected by lentiviral gene vectors according to one embodiment of the invention.

FIG. 6 reveals a morphology of a human liver tumor cell line ITRI-H16 cultured in ultra low attachment flask after 5 days, which is observed under a phase contrast microscope according to one embodiment of the invention.

FIG. 7 shows a relationship between tumor incidence and injection time, after implanting 100 ITRI-H16 cells and 1000 ITRI-H16 cells in sever combined immune deficient mice according to one embodiment of the invention.

### DETAILED DESCRIPTION OF DISCLOSED EMBODIMENTS

Below, exemplary embodiments will be described in detail with reference to accompanying drawings so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

In the disclosure, a human liver tumor cell line is cultured after isolating from a liver tumor tissue, and which is named as ITRI-H16. The ITRI-H16 cell line was deposited in the Food Industry Research and Development Institute with an accession number BCRC960432 on Nov. 7, 2011. According to a cell appearance morphology, a growth curve, an isoenzymes analysis, a genotyping, a cytogenic analysis, secreted proteins, and a secreted enzyme activity of the ITRI-H16 cell line, it shows that the human liver tumor cell line is, indeed, derived from a liver tissue of a HCV-related HCC patient. The human liver tumor cell line has tumor cell properties and is a newly established cell line. A result of a cancer stem cell property analysis reveals that the ITRI-H16 cell line highly expresses a cancer stem cell molecules, is capable of forming a spheroid structure, and is tumorigenic to the sever combined immune deficient mice, which indicates that the ITRI-H16 has a cancer stem cell potential. Furthermore, a result of an agent screening reveals that the established human liver tumor cell line ITRI-H16 has a susceptibility to cancer drugs, which is applicable in a cancer drug screening. In addition, an established luciferase expression system is employed for performing an in vivo image observation through an in vivo imaging system (IVIS) during a construction of a mice xenograft model. For drug developments and efficacy evaluations of liver cancer, the disclosure simultaneously provides an in vivo analysis platform and an in vitro analysis platform that can use in researches of a mechanism of hepatitis C viral carcinogenesis.

A method of establishing the human liver tumor cell line ITRT-H16 and relative testing methods thereof are described in detail as follows:

### Experiment 1

### Establishment of a Human Liver Tumor Cell Line ITRI-H16

In the disclosure, the human liver tumor cell line ITRI-H16 is cultured after isolating from a liver tumor tissue of a HCV-related HCC patient. The ITRI-H16 cell line was deposited in the Food Industry Research and Development Institute with an accession number BCRC960432 on Nov. 7, 2011.

After obtaining the liver tumor tissue from the HCV-related HCC patient, the tissue is immersed in Hank's balanced salt solution (HBSS). Next, the liver tumor tissue is cut into tumor tissue segments with a size of 5 mm*5 mm or smaller by a sterile scalpel. Then, under an cultured environment of 37° C., the liver tumor tissue segments are treated with a three-enzyme combined solution including dispase, collagenase, and DNase, so as to digest connective tissues, thereby releasing the liver cancer cells from the tissue at a lower damage level. Subsequently, a digestive solution containing the cells is filtered by a filter of a 100 um mesh. A cell suspension collected after filtering is transferred to a 50 ml centrifuged tube and is centrifuged at 1000 rpm for 5 minutes. A supernatant is removed. Next, 5 ml of red blood cell (RBC) lysis buffer is added and is reacted with the cell suspension for 3 minutes to remove red blood cells, and then the cell suspension is further centrifuged at 1000 rpm for 5 minutes to remove a supernatant. The cells are placed in 10 ml of a liver cell culture medium (Xenotech, K2300) and are cultured in a cell culture chamber of 5% CO₂ at 37° C. for primary culture.

Subculture

When a bottom of a cell culture plate is covered with the cells of the primary culture, an old culture medium is removed, and the plate is washed with PBS buffer. Then, trypsin is added to the cell culture plate for digestion, which decomposes attachment proteins between cell-cell or on sidewalls of the cell culture plate. Thus, the cells are detached from the sidewalls of the cell culture plate. By a ratio of 1:3-1:4 for the cells to the culture medium, a new culture medium is added for performing a subsequent culture. After that, the subculture is performed every four days so as to obtain the ITRI-H16 cell line having uniform morphology and higher purity.

Therefore, the human liver tumor cell line is successfully established, which the accession number thereof in the Food Industry Research and Development Institute is BCRC960432, and subsequent tests are performed. On the other hand, the human liver tumor cell line of the disclosure is not limited to the ITRI-H16 cell line described herein. A cell line is obtained by subcloning or monocloning derived from the ITRI-H16 cell line, or a cell line has any identifiable characteristics similar to the ITRI-H16 cell line, they fall within the scope of claims in the disclosure.

Particularly, in a process of a purification of the cancer cells, it often causes a large number of cells damaged. In addition, since the cancer cells are isolated from the tumor tissues, a growth of the cancer cells is significantly influenced by the mixed and mingled cells (i.e., lymphocytes, fibroblasts, tumor necrosis cells). It makes the isolation and the purification of the cancer cells quite difficult. Therefore, in the past, a success rate of the cancer cells in primary culture is low. In order to prevent that the other mixed and mingled non-cancer cells influence the cancer cell growth and further impact an establishment of the tumor cell line, we employs a serum-free medium. By removing a serum which is disadvantageous to a growth of fibroblasts in the culture medium, the serum-free medium is more beneficial for a growth of the cancer cells. Therefore, not only the doubts for having the mixed and mingled fibroblasts in the process of establishing the cancer cells are significantly reduced, but also the cancer cell growth is advantaged, therefore, the human tumor liver cell line ITRI-H16 is successfully established.

### Experiment 2

### Observation of a Cell Appearance Morphology of a Human Liver Tumor Cell Line ITRI-H16

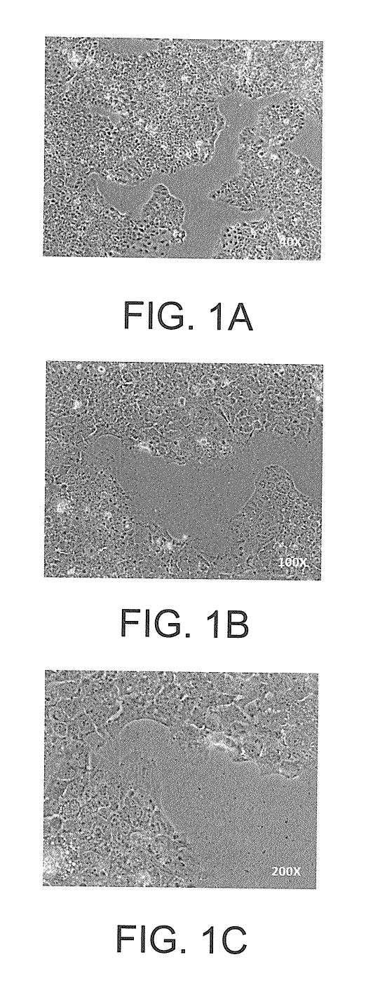

A human liver tumor cell line ITRI-H16 is placed under a phase contrast microscope (Nikon eclipse Ti-S) to observe the morphology with magnifications of 40×, 100×, and 200×, respectively, and results are shown in FIG. 1A to FIG. 1C.

Please refer to FIG. 1A to FIG. 1C, which reveals a morphology of a monolayer-cell of the human liver tumor cell line ITRI-H16, which is observed under the phase contrast microscope with magnifications of 40×, 100×, and 200×, respectively. According to FIG. 1A to FIG. 1C, the monolayer-cell of the human liver tumor cell line ITRI-H16 attaches to a culture plate coated with 1% collagen and shows the cell appearance of a larger karyoplasm ratio. When the cell confluency in culture plate is 80˜90%, a cell boundary between cell-cell becomes unclear, and the liver cells aggregates and arranged in a form similar to a hepatocyte island. The above characteristics indicate that the ITRI-H16 is classified in a moderated differentiation degree of the liver cancer cells.

### Experiment 3

### Determination of a Growth Curve of a Human Liver Tumor Cell Line ITRI-H16

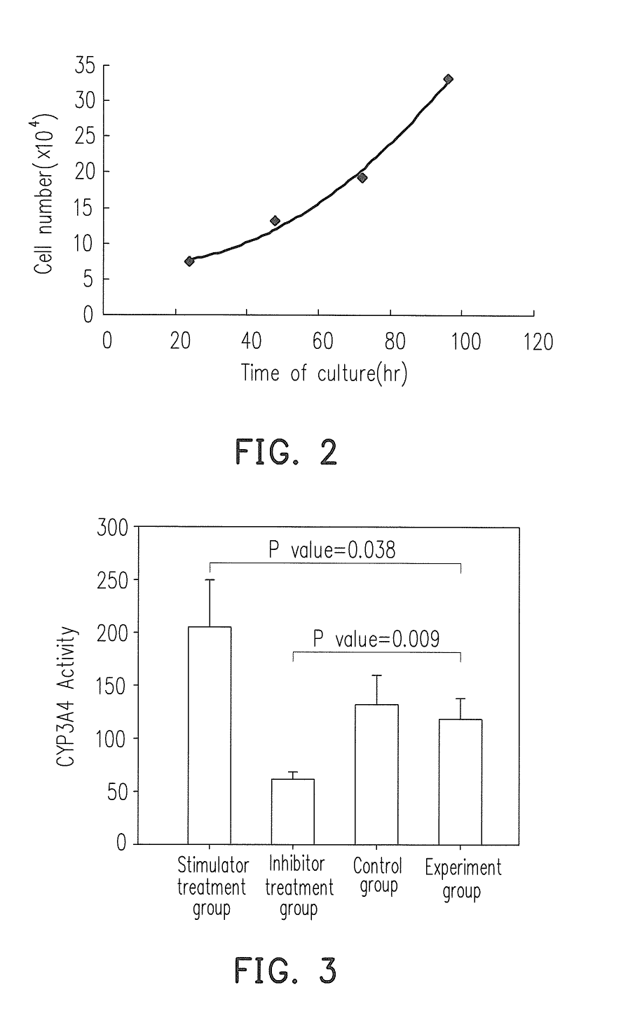

A test is used to determine a regularly growth curve of the human liver tumor cell line ITRI-H16, which is a continuously self-subculture of twelfth generation. 1×10⁵ of the ITRI-H16 cell lines are seeded in a 6-well plate, a serum-free medium (Xenotech, K2300) of liver cells is added to the plate, and a culture is performed in a constant-temperature cell culture chamber with 5% CO₂ at 37° C. Every 24-hour, the plate is taken from the chamber and is placed under a microscope for counting a cell number. A population doubling time of the cells is calculated and is shown in FIG. 2.

Please refer to FIG. 2, the population doubling time of the human liver tumor cell line ITRI-H16 is 34.0 hours. Namely, a growth speed rate of the ITRI-H16 cell line is rapid, which is one of the characteristics of the cancer cells. In addition, in the experiment, it is known that the serum-free medium is suitable to a long-term culture of the ITRI-H16 cell line.

### Experiment 4

### Isoenzyme Analysis of a Human Liver Tumor Cell Line ITRI-H16

In order to confirm the ITRI-H16 cell line is derived from humans without contaminating by other cells, the experiment employs AuthentiKit™ (Innovative Chemistry Marshfield, Mass.) and a manufacturer instruction thereof to perform isoenzyme analysis on seven isoenzymes of the ITRI-H16 cell line. Accordingly, corrected migration distances (CMD) of electrophoretic band of the isoenzymes are calculated. The seven isoenzymes are nucleoside phosphorylase (NP), glucose-6-phosphate dehydrogenase (G6PD), malate dehydrogenase (MD), mannose phosphate isomerase (MPI), peptidase B (PepB), aspartate aminotransferase (AST) and lactate dehydrogenase (LD). Meanwhile, a human 293 HEK cell line is treated as a human control group, and a porcine PK 15 cells line is treated as a porcine control group. General data of the electrophoretic band CMDs of the human isoenzymes serving as standard references is also provided, and results are shown in Table 1.

TABLE 1

| | Corrected Migration Distances (CMD) | | | | | | |
|---|---|---|---|---|---|---|---|
| Cell | NP | G6PD | MD | MPI | PepB | AST | LD |
| Porcine | 11.5 | 10.8 | 13.2 | 15 | 25.8 | 13 | 1.0 |
| control | | | | | | | |
| group PK | | | | | | | |
| 15 | | | | | | | |
| Human | 12.8 | 12.2 | 8.6 | 12 | 12.9 | 15.1 | −5.0 |
| control | | | | | | | |
| group 293 | | | | | | | |
| HEK | | | | | | | |
| Human | 12.9 | 14.5 | 8.3 | 12.9 | 12.4 | 15 | −5.6 |
| standard | | | | | | | |
| references | | | | | | | |
| ITRI- | 14 ± | 14.2 ± | 9.2 ± | 12.0 ± | 12.9 ± | 14.7 ± | −5.0 ± |
| H16 | 2 | 2 | 2 | 2 | 2 | 2 | 2 |

The electrophoretic band CMDs of the isoenzymes of the ITRI-H16 cell line, the 293 HEK cell line, and the PK 15 cell line are shown in Table 1. According to Table 1, by comparing zymography of the seven isoenzymes of the ITRI-H16 cell line, the 293 HEK cell line, and the PK 15 cell line, the ITRI-H16 cell line and the human 293 HEK cell line have similar zymography. In addition, the zymography of the ITRI-H16 cell line is significantly different from that of the PK 15 cell line. It can be seen that the ITRI-H16 cell line and the human 293 HEK cell line share the same origin. Namely, the ITRI-H16 cell line is classified in human cells.

### Experiment 5

### Genotyping of a Human Liver Tumor Cell Line ITRI-H16

In order to confirm the human liver tumor cell line ITRI-H16 is derived from a liver tumor tissue resected from a liver cancer patient, the experiment employs AmpFISTR Identifier PCR Amplification Kit to analyze the ITRI-H16 cell line DNA so as to perform a DNA fingerprinting confirmation of the ITRI-H16 cell line. In detail, fifteen short tandem repeats (SRT) of the ITRI-H16 cell line and segments of allelic pattern of a X-Y homologous gene (amelogenin) are analyzed. The fifteen SRTs of the ITRI-H16 cell line are D831179, D21S11, D7S820, CSF1PO, D3S1358, TH01, D13S317, D16S539, D2S1338, D19S433, vWA, TPDX, D18S51, D5S18, and FGA. Meanwhile, the same method is adopted to analyze allelic patterns of the patient's peripheral blood mononuclear cell (PBMC) and the resected liver tumor tissue in Experiment 1, and results are shown in Table 2.

TABLE 2

| | | Peripheral | | |
|---|---|---|---|---|
| | | blood mono- | Resected | ITRI-H16 |
| | | nuclear | liver tumor | cell line, |
| | Chromosomal | cell, allele | tissue, allele | allele |
| STR locus | Location | (1, 2) | (1, 2) | (1, 2) |
| D8S1179 | 8 | 14, 15 | 14, 15 | 14, 15 |
| D21S11 | 21q11.2-q21 | 29, 32.2 | 29, 32.2 | 29, 32.2 |
| D7S820 | 7q11.21-22 | 10 | 10 | 10 |
| CSF1PO | 5q33.3-34 | 10, 12 | 10, 12 | 10, 12 |
| D3S1358 | 3q | 15, 16 | 15, 16 | 15, 16 |
| TH01 | 11p15.5 | 8, 9 | 8, 9 | 8, 9 |
| D13S317 | 13q22-31 | 12 | 12 | 12 |
| D16S539 | 16q24-qter | 9, 11 | 9, 11 | 9, 11 |
| D2S1338 | 2q35-37.1 | 24 | 24 | 24 |
| D19S433 | 19q12-13.1 | 13 | 13 | 13 |
| vWA | 12q12-pter | 17, 18 | 17, 18 | 17 |
| TPOX | 2q23-2per | 8 | 8 | 8 |
| D18S51 | 18q21.3 | 15 | 15 | 15 |
| D5S818 | 5q21-31 | 11, 12 | 11, 12 | 11, 12 |
| FGA | 4q28 | 24, 26 | 24, 26 | 24, 26 |
| Amelogenin | X: p22.1-22.3 and | X, Y | X, Y | X |
| | Y: p11.2 | | | |

The Genotyping data of the ITRI-H16 cell line, the patient's peripheral blood mononuclear cell, and the resected liver tumor tissue are shown in Table 2. According to Table 2, the genotyping of the ITRI-H16 cell line, the patient's peripheral blood mononuclear cell, and the liver tumor tissue are identical, unique and consistent, where the STR-PCR pattern does not exist in the existing data library. Therefore, the ITRI-H16 cell line is, indeed, a novel cell line derived from the liver tumor tissue resected from the patient of liver cancer.

### Experiment 6

### Cytogenic Analysis of a Human Liver Tumor Cell Line ITRI-H16

The cytogenic analysis of the ITRI-H16 cell line is performed by the Center of Medical Genetics, Changhua Christian Hospital. In 20 cells examined, a variation of chromosome numbers is in a range of 60-90 (tetraploid), which reveals an abnormal chromosome number of the cells. Meanwhile, following abnormalities related to the chromosome numbers and structures are found in the observation. These abnormalities include (1) loss of chromosome Y, 1, 2, 3, 5, 6, 7, 8, 9, 10, 11, 12, 13, 14, 15, 16, 17, 18, 19, 20, 21, and 22; (2) isochromosomes of 8q, 14q, 17q, and 21q; (3) derivative chromosomes formed by an abnormal rearrangement of chromosomes including a derivative chromosome consisting a long arm of chromosome 11 and a long arm of chromosome 15, and a derivative chromosome consisting a long arm of chromosome 17 and a long arm of chromosome 21; (4) an additional material of an unknown origin on a short arm of chromosome 12; and (5) a presence of 1-8 marker chromosomes.

On the other hand, Spectral karyotyping probe kit (ASI, Inc.) is employed to perform a spectral karyotyping (SKY) analysis on the ITRI-H16 cell line. In 10 metaphase cells examined, chromosomal pattern of each of the cells is unique. Furthermore, consistent variations of karyotyping is reported as following: 66-72, XX, −X, −X, −Y, −Y, −Y, +1, der (3), +3, +5, +6, +7, der (8), +8, −11, der(12)t(Y; 12)×2, +12, −13, der(14), der(15), der(17)t(17; 21), +der(17)t(10; 17), −19, der(20)×3, +20, −21, and −22 [cp 10].

According to the above experimental results, abnormal separations of the chromosomes and multiple sets of the chromosomes reveal that the human liver tumor cell line ITRI-H16 has the cancer cell properties.

### Experiment 7

### Function of Secreting Proteins of a Human Liver Tumor Cell Line ITRI-H16

Retrospect to the clinical records of a HCV-related HCC patient which the ITRI-H16 cell line is derived from, high expression level of alpha-fetoprotein (APP) and anti-HCV antibodies in a serum of the patient is found. It indicates that the liver cancer patient had been infected with HCV. However, extracting RNA of a supernatant and a cell lysate of the ITRI-H16 cell line is used to perform a virus detection, no HCV RNA is detected. Namely, the current ITRI-H16 cell line carrying no hepatitis C virus is confirmed.

A secretion analysis experiment of AFP and albumin of the human liver tumor cell line ITRI-H16 is performed. In the experiment, 2×10⁵ ITRI-H16 cells are seeded in a 6-well plate, a serum-free medium (Xenotech, K2300) of liver cells is added to the plate, and a culture is performed in a constant-temperature cell culture chamber with 5% CO₂ at 37° C. Next, every 24-hour and 48-hour, the plate is taken from the chamber and is checked with the amount of AFP and albumin in the supernatant of the ITRI-H16 cell line. Meanwhile, a Huh-7 cell line having the same cell number is used as a control group. Results are shown in Table 3.

TABLE 3

| | Albumin (ng/ml) | | AFP(ng/ml) | |
|---|---|---|---|---|
| Cell | 24 hours | 48 hours | 24 hours | 48 hours |
| Huh-7 | 74.4 ± 1.7 | 157.0 ± 8.0 | 58.4 ± 0.1 | 63.3 ± 0.8 |
| ITRI-H16 | 328.4 ± 54.6 | 451.3 ± 25.6 | 48.8 ± 0.4 | 53.7 ± 1.4 |

The secreted amount of albumin and AFP of the ITRI-H16 cell line and the Huh-7 cell line are shown in Table 3. According to Table 3, the high expression level of AFP is still found after the ITRI-H16 cell line is cultured in vitro for a time period. Furthermore, since albumin is synthesized by the liver cells, it is known that the ITRI-H16 cell line has functions of synthesis and release of albumin. According to the above results, under a particular serum-free culture environment, the ITRI-H16 cell line is capable of maintaining similar functions compared to the functions of expressing AFP and albumin in the HCV-related HCC patient.

### Experiment 8

### Function of Secreting CYP3A4 of a Human Liver Tumor Cell Line ITRI-H16

CYP3A4 is a drug-metabolizing enzyme expressed by liver cells, which plays an important role in a drug metabolizing function of liver cells. Therefore, a secretion analysis experiment of CYP3A4 of the human liver tumor cell line ITRI-H16 is performed. In the experiment, 1×10⁵ ITRI-H16 cells are seeded in a 96-well plate, a serum-free medium (Xenotech, K2300) of liver cells is added to the plate, and a culture is performed in a constant-temperature cell culture chamber with 5% CO₂ at 37° C. The cells are divided into four groups. The first group is an untreated experiment group. The second group is a CYP3A4-specific stimulator treatment group by adding Rifampicin with a final concentration of 10 uM. The third group is a CYP3A4-specific inhibitor treatment group by adding Ketoconazole with a final concentration of 10 uM. The fourth group is a control group by adding dimethyl sulfoxide(DMSO) with a final concentration of 0.1%. After a 48-hour growth period for the ITRI-H16 cell lines of the above groups, a CYP3A4 activity is examined by a P450-GLO™ CYP3A4 assay, and results are shown in FIG. 3 and Table 4.

TABLE 4

| | Stimulator | Inhibitor | Control | |
|---|---|---|---|---|
| | treatment | treatment | group | Experi- |
| | group | group | (0.1% | mental |
| | (10 uM) | (10 uM) | DMSO) | group |
| ITRI-H16 cell | 205.0 ± 44.8 | 61.5 ± 6.8 | 132.1 ± 27.6 | 118.4 ± 19.7 |
| line | | | | |

Please refer to FIG. 3 and Table 4 for the CYP3A4 activity of the experiment group, the simulator treatment group, the inhibitor treatment group, and the control group of the human liver tumor cell lines ITRI-H16. According to FIG. 3 and Table 4, the ITRI-H16 cell line is capable of expressing CYP3A4, in which the CYP3A4 activity expressed by the ITRI-H16 cell line increases due to a stimulator and decreases due to a inhibitor. Namely, the expression of CYP3A4 by the ITRI-H16 cell line functions normally.

### Experiment 9

### Application of a Human Liver Tumor Cell Line ITRI-H16 on an Agent Screening

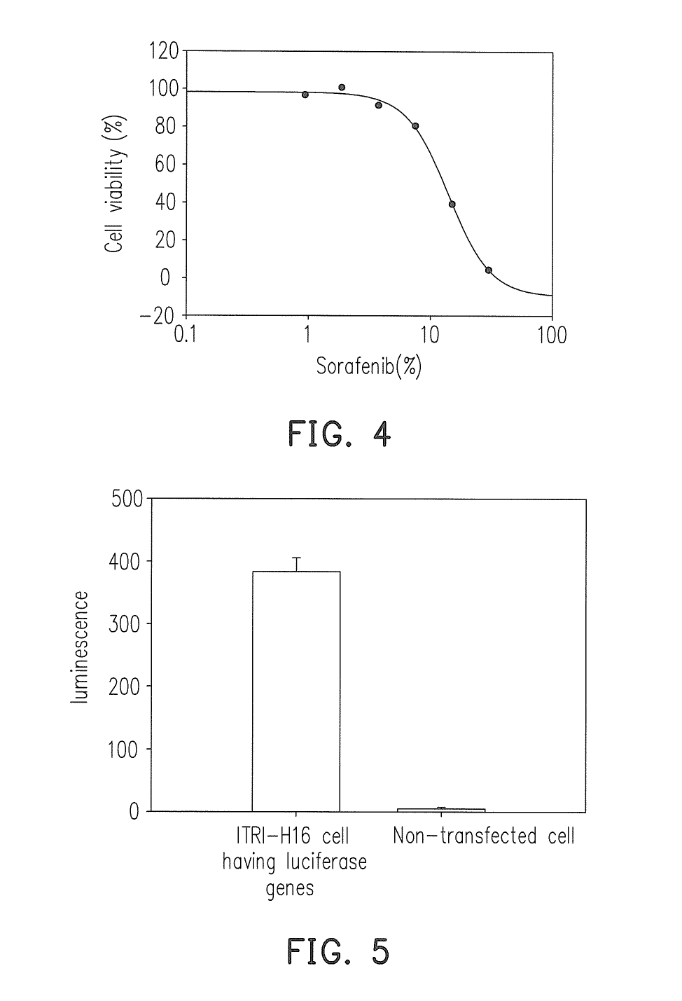

In the experiment, a plurality of plates are prepared with 1×10⁴ ITRI-H16 cells seeded respectively thereon and divided into a plurality of groups, and the groups are treated with different concentrations of Sorafenib, such as 30, 15, 7.5, 3.75, 1.88, and 0.94 uM. Next, after a 48-hour treating period, a cell viability of each of the groups is measured by an Alarmablue measurement assay, and results are shown in FIG. 4.

FIG. 4 shows the cell viability of the human liver tumor cell line ITRI-H16 in each of the groups after treating with Sorafenib in different concentrations. A calculation is done according to the results of FIG. 4, a half maximal inhibitory concentration (IC₅₀) of Sorafenib in the ITRI-H16 cell line are about 14.02±1.45, and a statistical P value is 0.01. According to the above results, the human liver tumor cell line ITRI-H16 has a susceptibility to the HCV-related HCC drug, which is able to use in a HCV-related HCC drug screening. Furthermore, according to the above experiments, the human liver tumor cell line ITRI-H16 has cancer cell properties, which can be used widely in a liver drug screening.

### Experiment 10

### Establishment of a Vector Expression System of a Human Liver Tumor Cell Line ITRI-H16

In the experiment, a 6-well plate seeded with 2×10⁵ of the fourth generation of the ITRI-H16 cell lines is prepared. Next, a transfection solution containing 5 ug/ml of polybrene is used as a culture medium. The transfection solution is added into the ITRI-H16 cells with a ratio of every 1000-cell to 1 ul-transfection solution (Firefly Luciferase (FLuc) Lentivirus with Puro Selection). The plate is placed in a constant-temperature cell culture chamber with 5% CO₂ at 37° C. for culture. After a 16-hour transfection, the transfection solution is removed. A cell culture medium is added, and puromycin with a concentration of 5 ug/ml is used for performing a screening. After subculturing four generations in the culture medium containing puromycin, a ONE-Glo™ Luciferase assay is performed to examine luminescence of the cells, and results are shown in FIG. 5. The luminescence signal of non-transfection cells is negligible low.

Please refer to FIG. 5, which shows luminescence measured in the human liver tumor cell line ITRI-H16 transfected by lentiviral gene-containing vectors. According to FIG. 5, an average luminescence of the ITRI-H16 cell line is 385.1±23.1. Namely, the ITRI-H16 cell line is capable of expressing luciferase genes. In the experiment, the established luciferase expression system is employed for performing an in vivo image observation through an in vivo imaging System (IVIS). For the drug developments and the efficacy evaluations of liver cancer, the disclosure provides an in vivo analysis platform.

### Experiment 11

### Expressing Cancer Stem Cell Molecules of a Human Liver Tumor Cell Line ITRI-H16

In the experiment, using markers specific for cancer stem cells as targets, the markers are CD13, CD24, CD44, CD133, EpCAM, and OV6. The markers on surfaces of the ITRI-H16 cells are dyed via an immunofluorescence staining. Next, a number of the dyed ITRI-H16 cells are detected by a flow cytometer so as to obtain a cell ratio (%) of the cells expressing specific markers to the total cells, and results are shown in Table 5 as below. The expression of albumin is treated as a control group.

TABLE 5

| | Ratio of cells expressing specific |
|---|---|
| Marker | markers to total cells, % |
| CD13 | 3.88 |
| CD24 | 65.9 |
| CD44 | 23.2 |
| CD133 | 0.12 |
| EpCAM | 0.35 |
| OV6 | 82.39 |
| Albumin | 77.36 |

A level of the cancer stem cell markers expressed on the surfaces of the ITRI-H16 cell line is shown in Table 5. In Table 5, CD 24 and OV 6 are expressed by 60% of the ITRI-H16 cells or more, and expressions of CD133 and EpCAM by the cells are not significant. According to the above results, the ITRI-H16 cell line has a cancer stem cell potential.

### Experiment 12

### Morphology of Cancer Stem Cells of a Human Liver Tumor Cell Line ITRI-H16

The cancer stem cells are a group of self-renewal cells existing in a tumor. Currently, the cancer stem cells not only play an important role in proliferation of the tumor, but also play an important role in treatment resistance and cancer recurrence. Generally speaking, a sphere formation of stem cells is a phenotype of the cancer stem cells and is evaluated.

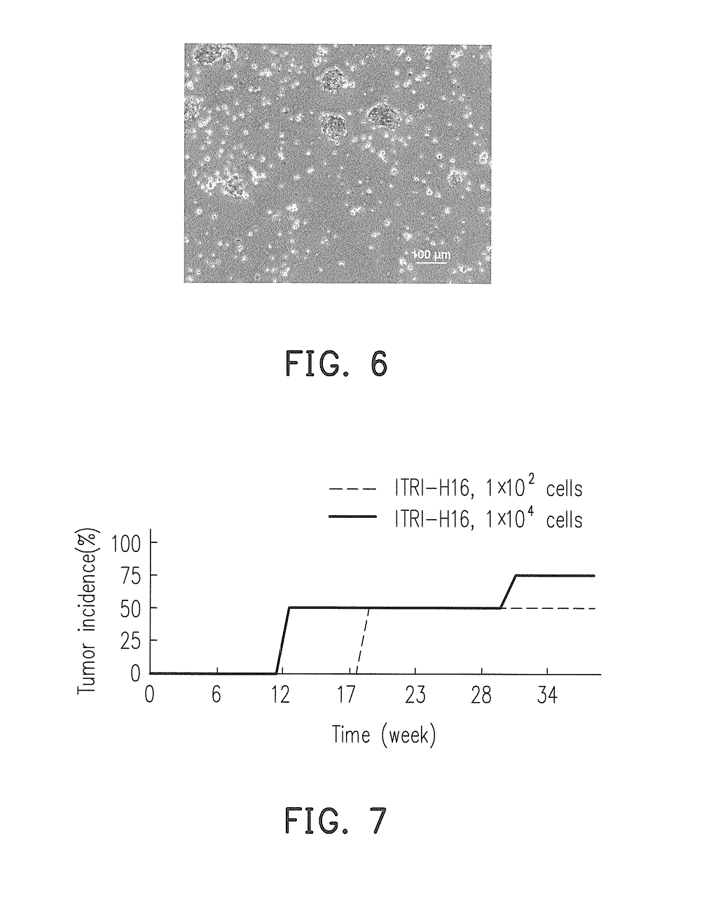

In the experiment, 2×10⁵ ITRI-H16 cell lines are seeded in a 6-well ultra low attachment flask, and a suspended culture is performed in a constant-temperature cell culture chamber with 5% CO₂ at 37° C. for 5 days. After a 5-day culturing period, the ITRI-H16 cell lines are placed under a phase contrast microscope (Nikon eclipse Ti-S) to observe cell morphology with magnification of 100×. The results are shown in FIG. 6.

FIG. 6 reveals the morphology of the human liver tumor cell line ITRI-H16 cultured in the ultra low attachment flask for 5 days, which is observed under the phase contrast microscope. According to FIG. 6, the ITRI-H16 cell line forms the spheroid-like structure of stem cell in the ultra low attachment flask. Therefore, the ITRI-H16 cell line has a cancer stem cell potential.

### Experiment 13

### Carcinogenicity of a Human Tumor Cell Line ITRI-H16 in Sever Combined Immune Deficiency Mice

First, sever combined immunodeficiency mice (SCID mice) are prepared, which are 6-8 weeks of female mice purchased from BioLASCO Taiwan (stock: CB17/Icr-Prkdcscid/CrlBltw). Next, 100 ITRI-H16 cells and 1000 ITRI-H16 cells are respectively injected subcutaneously into the SCID mice, in which the ITRI-H16 cells are the mixing cells of the second generation and the seventh generation. After injecting the ITRI-H16 cells to the SCID mice, a volume of subcutaneous tumor of the SCID mice is measured three times a week so as to estimate a tumor incidence in the SCID mice.

Please refer to FIG. 7, which shows a graph of a relationship between tumor incidence and injection time, after injecting 100 ITRI-H16 cells and 1000 ITRI-H16 cells into the SCID mice. Generally speaking, tumorigenicity is one of important evaluation criteria of the cancer stem cells. Namely, as a small amount of cancer cells is capable of forming a new tumor, it indicates that the cancer stem cells have the ability of self-renewal and proliferation. According to FIG. 7, when 10000 ITRI-H16 cells are injected into the SCID mice, the tumor incidence after 30 weeks is up to 75%. When 100 ITRI-H16 cells are injected into the SCID mice, the tumor incidence after 12 weeks is up to 50%. It can be seen that the ITRI-H16 cells has high tumorgenicity which belongs to the cancer stem cell potential.

Through the above experimental results, the ITRI-H16 cell line is successfully isolated under a particular isolation condition, and which is cultured under a special culture condition of the uses of the serum-free medium, so the ITRI-H16 cell line is capable of continuously, stably culturing and subculturing in vitro. The characteristics and functions of the liver cancer cell are retained, for instances, productions of alpha-fetoprotein and expressions of albumin and CYP3A4 activity of the liver cells. In addition, the ITRI-H16 cell line has the morphology of the cancer stem cells. A result of immunity analysis indicates that the ITRI-H16 cell line expresses the cancer stem cell markers, CD24 and CD44. Particularly, by analyzing the carcinogenicity of the ITRI-H16 cell line to SCID mice, it is found that the small amount of the ITRI-H16 cells has the ability to induce tumor growth in the mice. In other words, the ITRI-H16 cell line has a higher degree of carcinogenicity than other liver caner cell lines (such as Huh-7, PCL/PRF/5, HepG2, and Hep3B). It shows the ITRI-H16 cell line is a cell line having the cancer stem cell potential. On the other hand, for the drug developments, the ITRI-H16 cell line provides an in vitro analysis platform for evaluating the therapeutic efficacy HCV-related HCC drug. According to the above characteristics of the ITRI-H16 cell line, the ITRI-H16 cell line is also suitable for applications on a drug screening of drugs for liver failure and liver cancer stem cell.

The ITRI-H16 cell line is an established liver tumor cell line derived from the tissue of the HCV-related HCC patient, which can make up the deficiency of a drug development platform of only HBV-related or HBV-negative liver cancer. The ITRI-H16 is expected to play an important role in the field of HCV-related HCC research. By understanding the physiological characteristics of the IRTI-H16 cell line and the molecular-medicine characteristics thereof through the series of analyses as above, it is know that the ITRI-H16 cell line is capable of being used widely in the researches of mechanism of hepatitis C viral carcinogenesis, screening drugs for liver failure and liver cancer stem cell, liver cancer drug development, drug development for liver cancer stem cell, and drug metabolism.

In summary, the human liver tumor cell line of the disclosure is a first established cell research material derived from the tissue of the HCV-related HCC patient, and which is used as the research platform of the mechanism of HCV-related HCC and the drug screening related to liver cancer. In detail, the physiological properties and the molecular medicine characteristics of the IRTI-H16 cell line are studied in the series of analyses in above, which includes that the ITRI-H16 cell line is capable of being cultured in the serum-free medium thereby continuously and stably proliferating and subculturing, and having the cancer stem cell potential. Therefore, the ITRI-H16 cell line is expected to be used widely in the field of HCV-related HCC research so as to improve the chance of cure for HCV-related HCC.

Base on the above, the isolated human liver tumor cell line of the disclosure is the cell line isolated from the tissue of the HCV-related HCC patient, and which has the liver cancer cell properties and the cancer stem cell potential. Therefore, the isolated human liver tumor cell line of the disclosure is the suitable research material of the mechanism of HCV-related HCC, and which is used as a drug screening platform of liver cancer or liver failure.

It will be apparent to those skilled in the art that various modifications and variations can be made to the structure of the disclosure without departing from the scope or spirit of the disclosure. In view of the foregoing, it is intended that the disclosure cover modifications and variations of this disclosure provided they fall within the scope of the following claims and their equivalents.

### Biological Material Deposit

An isolated human liver tumor cell line of the disclosure is named as ITRI-H16. The ITRI-H16 cell line was deposited in the Food Industry Research and Development Institute with an accession number BCRC960432 on Nov. 7, 2011.

What is claimed is:

1. An isolated human liver tumor cell line, named as ITRI-H16.

2. The isolated human liver tumor cell line as recited in claim 1, wherein the ITRI-H16 cell line is established from a liver tumor tissue of a hepatitis C virus-related hepatocellular carcinoma (HCV-related HCC) patient, and a serum of the HCV-related HCC patient contains anti-HCV antibodies.

3. The isolated human liver tumor cell line as recited in claim 1, wherein the ITRI-H16 cell line secretes alpha-fetoprotein and albumin.

4. The isolated human liver tumor cell line as recited in claim 1, wherein the ITRI-H16 cell line has a cancer stem cell potential.

5. The isolated human liver tumor cell line as recited in claim 1, wherein the ITRI-H16 cell line expresses CD24 and OV6.

6. The isolated human liver tumor cell line as recited in claim 1, wherein the ITRI-H16 cell line forms a spheroid-like structure of stem cell in an ultra low attachment flask.

7. The isolated human liver tumor cell line as recited in claim 1, wherein the ITRI-H16 cell line has carcinogenicity in sever combined immunodeficiency (SCID) mice.

8. The isolated human liver tumor cell line as recited in claim 1, wherein the ITRI-H16 cell line grows and proliferates in a serum-free medium.

9. The isolated human liver tumor cell line as recited in claim 8, wherein the serum-free medium is a serum-free medium of liver cells (Xenotech, 1(2300).

10. The isolated human liver tumor cell line as recited in claim 1, wherein the ITRI-H16 cell line further comprises a reporter gene.

11. The isolated human liver tumor cell line as recited in claim 10, wherein the reporter gene expresses fluorescence or luminescence.

12. A method of an agent screening, comprising:
providing an isolated human liver tumor cell line, named as ITRI-H16 cell line; and treating an interesting compound to the isolated human liver tumor cell line so as to determine an effect of the interesting compound on the isolated human liver tumor cell line.

13. The method of an agent screening as recited in claim 12, wherein the interesting compound comprises a hepatitis C virus-related hepatocellular carcinoma (HCV-related HCC) drug.

14. The method of an agent screening as recited in claim 12, wherein the interesting compound comprises a drug for liver failure.

15. The method of an agent screening as recited in claim 12, wherein the interesting compound comprises a drug for liver cancer stem cell.

16. The method of an agent screening as recited in claim 12, wherein the effect of the interesting compound is determined by a half maximal inhibitory concentration (IC₅₀) for the isolated human liver tumor cell line.

17. The method of an agent screening as recited in claim 12, wherein the effect of the interesting compound is determined by a metabolism analysis of the isolated human liver tumor cell line.

18. The method of an agent screening as recited in claim 12, wherein the ITRI-H16 cell line further comprises a reporter gene.

19. The method of an agent screening as recited in claim 12, wherein the reporter gene expresses fluorescence or luminescence.

20. A cell line is obtained by subcloning or monocloning derived from the ITRI-H16 cell line.
